(19) United States
(12) Patent Application Publication
(10) Pub. No.: US 2023/0129156 A1
(43) Pub. Date: Apr. 27, 2023

(54) NON-INVASIVE THERMOMETRY APPARATUS

(71) Applicant: HYPERTHERMIA CANCER INSTITUTE LLC, Santa Monica, CA (US)

(72) Inventors: Henry Harrison LANDAU, Santa Monica, CA (US); Cynthia KEPPEL, Norfolk, VA (US)

(73) Assignee: HYPERTHERMIA CANCER INSTITUTE LLC, Santa Monica, CA (US)

(21) Appl. No.: 17/973,444

(22) Filed: Oct. 25, 2022

Related U.S. Application Data
(60) Provisional application No. 63/271,372, filed on Oct. 25, 2021.
(60) Provisional application No. 63/271,358, filed on Oct. 25, 2021.

(51) Int. Cl. A61B 5/01; G01K 1/143; G01J 5/00; G01K 7/02; G01K 13/20; A61B 5/00

(52) U.S. Cl. CPC A61B 5/01; A61B 5/0008; G01J 5/0025; G01K 1/143; G01K 7/02; G01K 13/20; A61B 2562/0271; G01J 2005/0077

(57) ABSTRACT

A thermometry apparatus used during hyperthermia therapy, which has a mat that can be used in combination with a non-invasive thermometry system. The mat has a top face and a bottom face. Between the top face and the bottom face are embedded wires. The wires provide skin and treatment head thermal information based on the thermal coefficient of resistance of the wires or via two metals in a thermocouple configuration. The mat is placed between the skin and an ultrasound head. The mat is flexible enough to conform to the patient’s body shape at the treatment point. The mat may be used in combination with an infrared camera, where at least one IR camera is pointed at a semi perpendicular angle to the mat, whereby the IR camera measures the temperature directly from the mat side and continually below the ultrasound head providing thermal depth measurements.

### CROSS-REFERENCE TO RELATED APPLICATIONS

This application is a non-provisional of, and claims priority to, U.S. Provisional Pat. Application Number 63/271,358 filed on Oct. 25, 2021, as well as, U.S. Provisional Pat. Application No. 63/271,372 also filed on Oct. 25, 2021; which applications are incorporated herein by reference.

### STATEMENT REGARDING FEDERALLY SPONSORED-RESEARCH OR DEVELOPMENT

None.

### FIELD OF THE INVENTION

The present invention generally relates to thermometry, and more particularly relates to a non-invasive thermometry apparatus for use during hyperthermia therapy.

### BACKGROUND

Hyperthermia therapy is the use of heat to enhance other cancer treatment methods such as chemotherapy. The temperature at which a tumor is heated is of ultimate importance, however, that needs to be mitigated by not delivering uncomfortable temperature to the patient. Optimally, hyperthermia therapy would heat only an area with a tumor. Since most tumors are sub-dermal (within a body), ultrasound has been found to be an effective method of heating the tumor.

Using specialized crystals, focused ultrasound waves can be generated and directed to heat tumors at depth while heating surrounding areas far less than many other methods. Ultrasound heats by depositing energy where the ultrasound waves are absorbed. Ultrasound, like any sound, requires energy to be created and that energy is carried with the wave. When the wave is absorbed, by the tumor for instance, the wave stops along with its energy. The energy that previously made up the ultrasound becomes heat where the wave is absorbed.

The reason why ultrasound is so effective at heating only the directed area is its penetrative power. Tumors are very rarely at surface level and thus heating with no penetration would not be able to reach those tumors. While direct heat application can only directly heat the skin, ultrasound can penetrate a few centimeters below the skin to deliver its heat payload.

However, ultrasound is not perfect because most of the energy is actually absorbed before the full depth is reached, heating up the skin in the process. This is remedied using a cool water sac, or bolus, applied at the heating location. As the skin and tumor heat up, the bolus cools the skin to more comfortable temperatures by conducting heat away through the water.

The challenge with this process is the lack of thermal treatment delivery information provided to those operating the ultrasound equipment. Optimally, the hottest location will be the tumor and all locations will be below damage or discomfort temperatures. How does a clinical operator monitor the temperature at the tumor depth within the body? Current clinical thermometry used during hyperthermia treatments for cancer involves either (a) adhering individual sensors to the skin of a patient, which is an outdated, inefficient and uncomfortable process not easily duplicated between treatment sessions, or (b) utilizing sub-dermal needles at tumor depth, which is inefficient, invasive, and painful to the patient.

Measuring surface skin temperatures requires thin non-metallic probes, as metal can stop the ultrasound waves from penetrating the skin. Other known methods employ an array of thermocouples stuck to the patient directly during each treatment with sonogram gel for a measurement at the skin surface.

Currently, while there exists different apparatus and methods for the measurement of heat energy delivered to a sub-dermal tumor, there is a need in the art for an apparatus adapted for use in hyperthermia therapy that complements hyperthermia therapy treatments and can non-invasively provide temperature data to the clinician administering the hyperthermia therapy treatment. There is also a need in the art for an apparatus that improves the efficiency, repeatability, and resolution of the thermometry with the aim of improving patient comfort and reducing non-treatment time spent in the overall treatment process of hyperthermia therapy. Additionally, the current state of the art does not address the monitoring of temperature both at the treatment head surface and at the skin in order to provide thermal gradient information that differentiates treatment head and skin temperatures.

### SUMMARY

The invention disclosed herein includes an apparatus for thermometry that is preferably adapted for and complements hyperthermia therapy treatments and non-invasively provides real-time temperature data to the clinician administering the hyperthermia therapy treatment. Additionally, the invention improves the efficiency, repeatability, and resolution of the thermometry with the aim of improving patient comfort and reducing non-treatment time spent in the overall treatment process of hyperthermia therapy. Furthermore, the invention is configured to provide thermal gradient information that differentiates treatment head and skin temperatures, in a non-invasive manner. Disclosed herein, a non-invasive thermometry apparatus is, generally, a mat, having a top surface, a bottom surface, and a thermally reactive layer captured between the top surface and the bottom surface. It is contemplated to be within the scope of the invention that the apparatus may also be configured to include a plurality of discreet thermally reactive layers, with a separator between each layer. The non-invasive thermometry apparatus is further configured to output, display, or otherwise, transmit thermal data relative to the reaction of the thermal layer, or layers, to a thermal flux via a bus (in the context of this disclosure, a bus refers to a structural component of the apparatus configured to facilitate the acquisition of the thermal data, such as, but not limited to: exposed wires, electrical contacts, solder points, optical fibers, and other data acquisition and transfer structures).

One non-limiting embodiment of the present invention is a sandwiched composite having a top surface, a bottom surface, and a plurality of thermally reactive filaments captured between the top surface and the bottom surface. The top and bottom surfaces are substantially thermally transparent. Meaning that, the top and bottom surfaces are made of materials that, while capturing and constraining the plurality of thermally reactive filaments, do not block or absorb any (or nearly any) of the heat energy to be delivered to the treatment location.

By way of example, and not limitation, an embodiment of the apparatus configured for use in hyperthermia therapy treatment, where the heat treatment is delivered via ultrasound wave energy, the apparatus would include a grid of thermally reactive filaments captured between a top surface and a bottom surface. Here, the material of the top and bottom surface are chosen of a material that will not substantially block nor absorb the ultrasound waves that pass from an ultrasound treatment head through the apparatus and into the treatment area (a patient body part, for example).

In a preferred embodiment, the apparatus is comprised of a top face, a bottom face, a middle wall between the top face and the bottom face, a first grid between the top face and middle wall, and a second grid between the middle wall and the bottom face.

The apparatus is thus configured to provide an improvement over the current state of clinical thermometry in that the apparatus provides an easy, repeatable and non-invasive structure that is ideally suited for use in hyperthermia therapy treatments.

It is one of the main objects of the present invention to provide a non-invasive thermometry apparatus for use during hyperthermia therapy.

It is another object of this invention to provide a thermometry apparatus used during hyperthermia therapy that comprises a mat between an ultrasound head and the skin of a patient.

It is another object of this invention to provide a thermometry apparatus for use during hyperthermia therapy comprising a mat having thermocouple and/or resistive wires embedded therein.

It is another object of this invention to provide a thermometry apparatus for use during hyperthermia therapy, which has a mat having an embedded fixed and repeatable thermal sensor geometry.

It is another object of this invention to provide a thermometry apparatus used during hyperthermia therapy that is associated with a faster set-up time that what is currently known in the art.

It is another object of this invention to provide a thermometry apparatus for use during hyperthermia therapy that is less prone to human error.

It is another object of this invention to provide a thermometry apparatus for use during hyperthermia therapy that can provide digitized feedback to the treatment head controls and clinicians.

It is another object of this invention to provide a thermometry apparatus for use during hyperthermia therapy that measures the thermal gradient between treatment head and skin temperatures.

It is another object of this invention to provide a thermometry apparatus used during hyperthermia therapy that is easily sanitized for re-use between patient uses.

It is another object of this invention to provide a thermometry apparatus for use during hyperthermia therapy, which is flexible yet of a durable and reliable construction.

Other non-limiting embodiments of the present invention are contemplated and described below.

The present invention differs from the current state of the art in that the apparatus is non-invasive, repeatable, and easy to use. A cost effective and easy to implement thermometry device is one that is likely to be implemented and, therefore, can provide benefits both to the patient and the clinical user during hyperthermia therapy treatments.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a flowchart showing a prior art method of hyperthermia therapy;

FIG. 2 is an isometric view of a mat, according to an embodiment of the present invention;

FIG. 3 is an exploded view of the mat, according to an alternative embodiment of the present invention;

FIG. 4 is a schematic representation of a system incorporating the apparatus, according to an embodiment of the present invention;

FIG. 5 is an isometric representation of FIG. 4; and

FIG. 6 is a flowchart representing a method of use of the apparatus, according to an embodiment of the present invention.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

We disclose here an apparatus for thermometry adapted for use in hyperthermia therapy treatments. It will be understood that, although the description provided here is placed in the context of a hyperthermia therapy treatment utilizing ultrasound as the heating method, other applications of the apparatus are contemplated to be within the scope of the invention as may be apparent to a person ordinarily skilled in the art.

### The Apparatus

Referring now to the drawings, the present invention is a thermometry apparatus used during hyperthermia therapy, and is generally referred to with numeral 10. It can be observed that it basically includes mat 20, which can be used in conjunction with infrared camera 50.

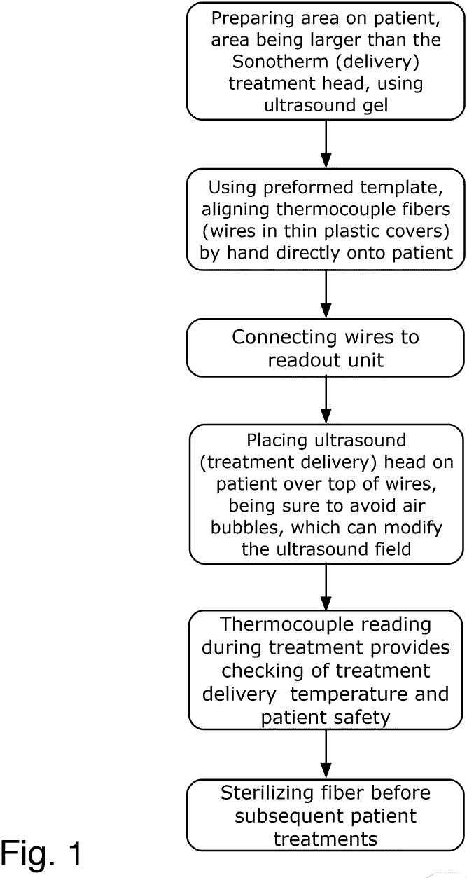

As seen in FIG. 1, according to the prior art, a current method for hyperthermia treatment comprises the following steps:
a) preparing an area on patient P using ultrasound gel;
b) aligning thermocouple fibers or wires in thin plastic covers by hand directly onto patient P;
c) connecting wires to a readout unit;
d) placing ultrasound head 40 on patient P over top of wires;
e) reading thermocouple during treatment, which provides checking of treatment delivery temperature and patient P safety; and
f) sanitizing sensing wires or fibers 26 after patient treatment.

The proposed technology according to embodiments of the invention, and as shown in, for example, mat 20, replaces the need to manually place and align the wires, saving time, awkwardness of touching patient P, and leading to enhanced reproducibility. It can also facilitate both measurement at treatment head and skin S, and the thermal gradient between them. The treatment head may be ultrasound head 40.

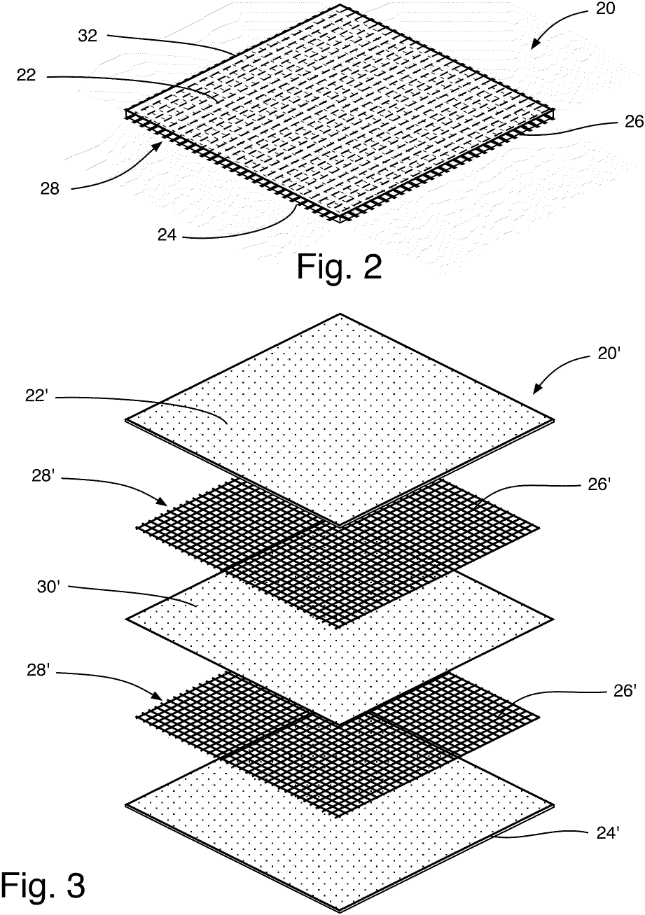

As seen in FIG. 2, mat 20 comprises top face 22 and bottom face 24. Between top face 22 and bottom face 24 are embedded wires 26 with a temperature sensitive resistivity calibratable to temperature. In a preferred embodiment, wires 26 are nickel wires. Wires 26, in other embodiments may be filaments chosen from other materials that have a thin cross section (approximately less than or equal to 0.75 mm) compared to length and are capable of measurably reacting to temperature variations.

Mat 20 is a thermometry mat comprising embedded wires 26 to provide thermal information based on the thermal coefficient of resistance (TCR) of wires 26. Wires 26 may be a single strand or multiple strands. Wires 26 may be arranged between the top face 22 and bottom face 24 in the form of a grid 28, and in a preferred embodiment, comprises at least one grid 28.

As metal is not “ultrasound transparent”, wires 26 that are made from a metallic material cannot cover too much the area under ultrasound head 40. In a preferred embodiment, therefore, wires 26 are spaced to allow approximately between 8 to 12 mm between wires. In a more referred embodiment, wires 26 are 10 mm apart to reduce coverage.

The overall shape of Mat 20 may be square, rectangular, or any other shape that is adapted to 1) adequately constrain the wires 26, and 2) remain placed in location during use. In a preferred embodiment, mat 20 is configured to be approximately the size of the ultrasound (US) head 40. This sizing benefits for alignment of the mat 20 to the desired treatment area. Mat 20 is placed between the skin S and ultrasound head 40. The material for the top face 22 and bottom face 24 of mat 20 is chosen such as to be flexible enough to conform to the patient’s body shape at the treatment point and should be ultrasound transparent, or similar to human tissue, for purposes of ultrasound transmission.

In a preferred embodiment, top face 22 and bottom face 24 of mat 20 is made of High Density Poly Ethylene (HDPE). High Density Poly Ethylene (HDPE) provides a good seal around wires 26 at edges 32 of mat 20, while not compromising the flexibility, cleaning, and durability requirements. The top face 22 and bottom face 24 of mat 20 may be applied to the patient with ultrasound gel (for ultrasound transmission without air pockets) that can be easily cleaned from the mat after.

Although, for purposes of compact disclosure, the apparatus is discussed in the context of ultrasound hyperthermia therapy, it should be evident to a person ordinarily skilled in the art that the apparatus can be configured to work with other heat energy types (such as, but not limited to, microwaves).

Referring now to FIG. 3, we discuss a first alternative embodiment of mat 20 defined as mat 20′. Mat 20′ comprises top face 22′, bottom face 24′, and middle wall 30′. Mat 20′ further comprises first and second grids 28′. Middle wall 30′ may be placed between first and second grids 28′. First and second grids 28′ are parallel to each other inside mat 20′. Mat 20′ may be defined as a double mat.

Hyperthermia treatment requires monitoring the temperature of ultrasound head 40, skin S, and tumor T. In an improvement over the current state of the art, mat 20′ may be added to hyperthermia therapy systems to accomplish the monitoring of the temperature of ultrasound head 40 and skin S at the same time.

First grid 28′ is placed between top face 22′ and middle wall 30′ closer to ultrasound head 40, and second grid 28′ is placed between middle wall 30′ and bottom face 24′ closer to skin S, whereby first grid 28′ monitors the temperature of ultrasound head 40 and second grid 28′ monitors the temperature of skin S, as well as, the temperature gradient between them. First and second grids 28′ are made of wires 26′. Wires 26′ are made of nickel or other appropriate metal to provide thermal information based on the thermal coefficient of resistance (TCR) of wires 26′, as discussed above.

In a second alternate embodiment, mat 20 comprises sensors in a fixed array by embedding the sensors in a material.

In a third alternate embodiment, mat 20 comprises a hybrid of electric current resistive-wire and current-sensors. Mat 20 may also have embedded miniature thermocouples, whereby each wire 26 is made of, for example, copper, within mat 20 from one side, and constantan or similar coming out of mat 20 from an opposite side, has a solder bead joining the copper and constantan as a location where the temperature is measured from a thermocouple.

In a fourth alternate embodiment, mat 20 is configured to constrain gel containing thermochromic pigment. The mat 20, in this embodiment, may be divided into small cells and surrounded by a flexible, durable, cleanable plastic casing that can have ultrasound gel applied and cleansed from it. Mat 20 may further have sensors, which measure the color of the gel in a cell by e.g. measuring the absorption of a specific wavelength through the thickness of gel in that cell. This would require that the gel remains viable across many heating/cooling cycles, and has highly temperature-dependent color in the range approximately between 36 and 46° C. with the ability to indicate temperatures outside that range. The color is calibrated to a temperature and so can be read visually by the clinician with access to a color-temperature conversion chart. Alternatively, or in addition, the gel color can be obtained via a digital camera or similar device and the information sent to a computer for comparison of stored wavelength to temperature information and the color (wavelength) from the gel.

In all embodiments, the apparatus includes structures whereby temperature data can be obtained. As such, a non-invasive thermometry apparatus according to embodiments of the invention is further configured to output, display, or otherwise, transmit thermal data relative to the reaction of the thermal layer, or layers, to a thermal flux via a bus (in the context of this disclosure, a bus refers to a structural component of the apparatus configured to facilitate the acquisition of the thermal data, such as, but not limited to: exposed wires, electrical contacts, solder points, and other data acquisition and transfer structures). In embodiments, the apparatus may be further configured to transmit the temperature data either via a wired or wireless connection to a system configured to receive the temperature data.

### The Apparatus in a Hyperthermia Treatment System:

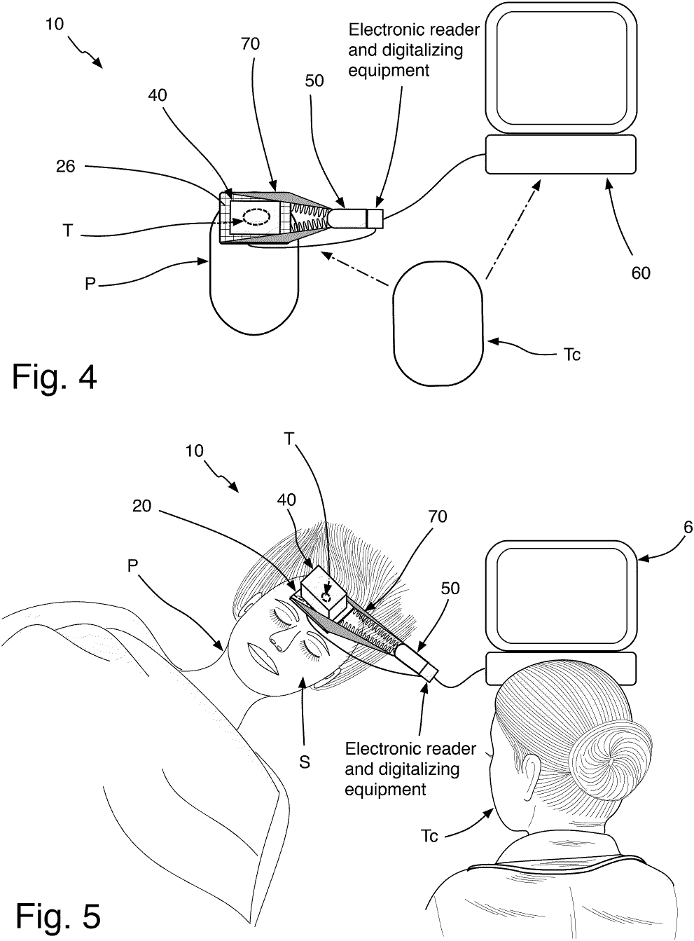

Referring now to FIGS. 4 and 5, mat 20, or mat 20′, seen in FIG. 3, is connected to an electronic reader and digitalizing equipment that is connected to computer device 60. In a preferred embodiment, mat 20 and mat 20′ are used in conjunction with an infrared camera 50. Mats 20 and 20′ can measure precisely and directly, but cannot assess anything about the delivery at tumor T. Mats 20 and 20′, coupled with infrared camera 50 improve repeatability and comfort of patient P while reducing room time per patient P. As shown in FIGS. 4 and 5, system 10 also has improved resolution over current individually placed sensors.

Mat 20 or mat 20′ for skin S and ultrasound head 40 have benefits associated with a faster set-up time, as compared to a plurality, as many as sixteen as is known in the art, thermocouples being individually placed with gel on patient P. In addition, mat 20 and mat 20 provide the following advantages:
fixed, repeatable geometry
less prone to human error
easier and faster to clean
provide digitized feedback to the treatment head controls and the clinician
may be used in conjunction with IR camera 50

### A Method of Using the Apparatus:

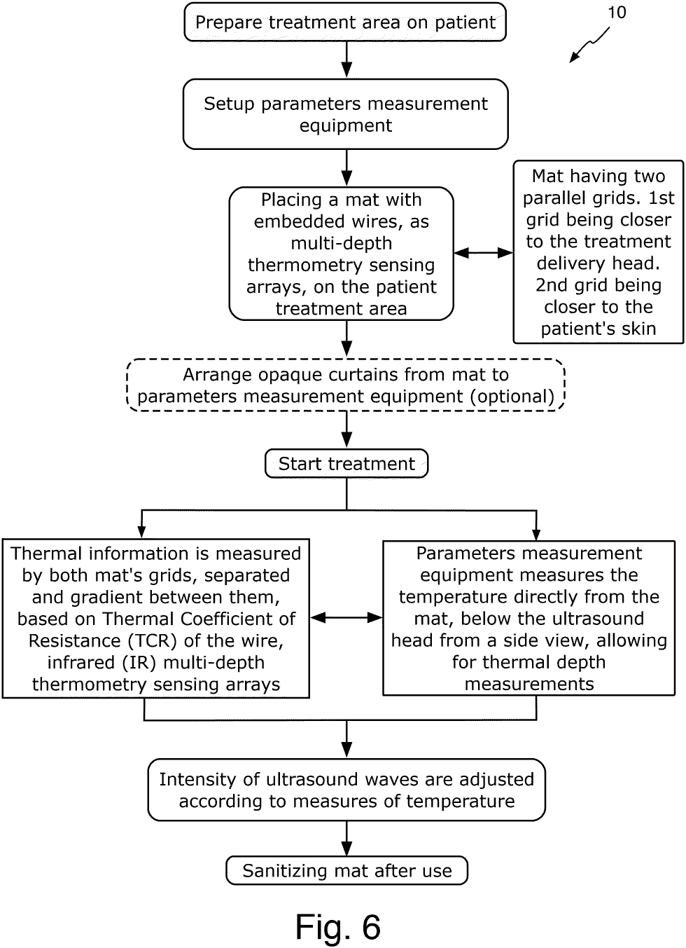

Referring now to FIG. 6, a method of using embodiments of the disclosed apparatus in a non-invasive thermometry system used during Hyperthermia Treatment Therapy comprises the following steps:
preparing a treatment area on patient P;
setting up at least one IR camera 50 to provide 2D or 3D temperature measurements, two or more cameras 50 are placed at a predetermined angle;
placing mat 20, or mat 20′, on patient P treatment area;
arranging curtains 70 from mat 20 or mat 20′ to IR camera 50;
placing ultrasound head 40 over mat 20 or mat 20′;
starting treatment, taking mat 20 or mat 20′ temperatures, as well as thermal field size and shape measurements from IR camera 50, and adjusting ultrasound waves intensity according to the temperature or temperature gradient measurements; and
sanitizing mat 20 and mat 20′ after patient use.

The step of arranging curtains 70 from mat 20 or mat 20′ to IR camera 50 is optional.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, although do not preclude the presence or addition of one or more other features, integers, steps, operations, elements, components, and/or groups thereof.

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand various embodiments of the present invention, with various modifications as are suited to the particular use contemplated.

What is claimed is:

1. A non-invasive thermometry apparatus comprising:
a top surface;
a bottom surface;
a one or more thermally-reactive layer captured between the top surface and the bottom surface; and
a bus in operable connectivity with each of the one or more thermally-reactive layer.

2. The apparatus of claim 1 where the thermally-reactive layer is a grid of thermally-reactive wires.

3. The apparatus of claim 1 further comprising two or more thermally-reactive layers having a separator between each thermally-reactive layer.

4. The apparatus of claim 1 further comprising a data connection adapted to operably connect the bus to an external processor.

5. The apparatus of claim 4, wherein the data connection is a wireless data connection.
